(19) United States
(12) Patent Application Publication
(10) Pub. No.: US 2013/0164347 A1
(43) Pub. Date: Jun. 27, 2013

(54) MULTILAYER POLYMERIC DRUG DELIVERY SYSTEM

(71) Applicants: Michel Gensini, Flemington, NJ (US); Michael J. Trezza, II, Flemington, NJ (US); Abla Abu-Zayyad Creasey, Morristown, NJ (US)

(72) Inventors: Michel Gensini, Flemington, NJ (US); Michael J. Trezza, II, Flemington, NJ (US); Abla Abu-Zayyad Creasey, Morristown, NJ (US)

(21) Appl. No.: 13/724,483

(22) Filed: Dec. 21, 2012

Related U.S. Application Data
(60) Provisional application No. 61/579,350, filed on Dec. 22, 2011.

(51) Int. Cl. A61K 9/00

(52) U.S. Cl. 424/400

(57) ABSTRACT

The invention relates to a customizable, solvent-free sustained release medical device for wound closure, wound healing, and the sustained release of an active. The device of the invention is composed of at least a polymeric carrier layer and optionally an oxidized regenerated cellulose layer(s). Polymeric barrier layers can be added to modify the release profile (burst, time of release, etc.) of the selected bioactive agent(s).

### FIELD OF THE INVENTION

The invention relates to a customizable, solvent-free sustained release medical device for wound closure, wound healing, and the sustained release of an active.

### BACKGROUND OF THE INVENTION

Current methods of wound closure include sutures and staples to provide adequate wound support for the duration of wound healing. Both techniques involve additional trauma to the wound. In addition, when either of these methods is used to close wounds inside the body, especially when sealing or attaching organs containing fluids, for example, intestine, blood vessels, and lungs, there is potential for fluid leaks that cause complications and high morbidity rates.

Direct application of adhesives have also been proposed and used for wound closure, especially alpha-cyanoacrylates, but their use is limited by the biodegradability and biocompatibility of commercial existing compositions. Monomers of alpha-cyanoacrylates are extremely reactive, polymerizing rapidly in the presence of minute amounts of an initiator including moisture present in the air or on moist surfaces such as animal tissue.

M. Milbocker, T. Lutri et al., and J. John et al. describe the combination of the above wound closure methods using adhesive compositions and bandage-like dressings, but do not address application for use in internal organs, for which an absorbable mesh-like material and an absorbable adhesive composition is needed. There is extensive literature in the area of alpha-cyanoacrylates but only very little on truly absorbable cyanoacrylates (H. Liu).

Sustained release medical devices are known and commercially available. Eisai Inc. markets a polifeprosan 20 with carmustine implant sold under the trademark GLIADEL® Wafer for the treatment of high-grade glioma and glioblastoma. (Up to 8 wafers can be implanted in the brain post resection.) R. Liu et al. teach that paclitaxel loaded flexible polymeric films (Poly (glycerol monostearate co-e-caprolactone)) can be used to treat non-small cell lung cancer (NSCLC), and F. Qian et al. discuss 5-Fluoro-Uracil (5-FU) loaded poly (lactic-co-glycolic acid) (PLGA) millirods for intratumoral drug delivery potentially applicable to liver cancer. See R. Liu et al., Ann. Surg Oncol 17:1203-1213, 2010 and F. Qian et al., J. Biomed Mater Res 61:203-211, 2002.

Oxidized regenerated cellulose (ORC) is well known in the medical field as a hemostatic agent or adhesion prevention material. ORC has also been combined with alpha-cyanoacrylates to provide tissue approximation devices incorporating tissue adhesives. See U.S. patent application Ser. No. 12/645,164, published on Jun. 23, 2011, as Patent Publication No. 2011/0152924 A1, incorporated herein by reference. ORC has never, however, been incorporated into a multilayer medical device wherein the medical device has a customizable release profile.

While the known and commercially available sustained release drug devices address the release of a single drug, they do not contemplate the release of a wide variety of classes of molecules. Furthermore, these sustained release drug devices do not address the issue of hemostasis. For instance, in the case of the polifeprosan 20 with carmustine implant sold under the trademark GLIADEL® Wafer, up to eight wafers have to be placed in the periphery of the wound, and then the wafers are typically covered with an absorbable hemostat such as the woven fabric sold under the trademark SURGICEL® ORIGINAL™ (available from Johnson & Johnson Wound Management, a division of Ethicon, Inc., Somerville, N.J.) to achieve hemostasis. These sustained release drug devices also do not address customizable release profiles, they do not allow for bimodal release profiles, and they cannot increase the update of drugs by tumors. For instance, millirods have water soluble plasticizers in the outer layer that limits the potential for customizing the size of the burst. Moreover, the drug loading of these sustained release drug devices is limited at 30 percent w/w in part because of the casting process (under moderate pressure: 4.6 MPa). Solvent based systems are challenging when there is a need to release more than one compound because of differences of solubility of the selected compounds. There is a need in this art for novel sustained release systems for use in internal wound closure, such as the gastrointestinal (GI) track, and healing as well as sustained release of a bioactive agent.

There is a need in this art for novel sustained release systems for use in internal wound closure, such as the gastrointestinal track, and healing as well as sustained release of a bioactive agent(s) for oncology as well as additional unmet medical needs.

### SUMMARY OF THE INVENTION

Solvent-free sustained release devices are disclosed. The devices comprise at least a carrier layer comprising a bioactive agent(s) and a first oxidized regenerated cellulose layer laminated to the upper surface of the carrier layer. In another embodiment, the device comprises a second oxidized regenerated cellulose layer laminated to the lower surface of the carrier layer. In yet another embodiment, the second oxidized regenerated cellulose layer is dry, partially neutralized to a pH of from about 5 to about 7 and the second oxidized regenerated cellulose layer further comprises a bioabsorbable alpha-cyanoacrylate adhesive composition.

In yet another embodiment, the devices comprise a carrier layer comprising a bioactive agent(s), a first and second barrier layer, and a first oxidized regenerated cellulose layer. The first barrier layer is laminated to the upper surface of the carrier layer, and the second barrier layer is laminated to the lower surface of the carrier layer. The first oxidized regenerated cellulose layer is laminated to the upper surface of the barrier layer. This embodiment can further comprise a second oxidized regenerated cellulose layer laminated to the lower surface of the second barrier layer. This second oxidized regenerated cellulose layer can be dry, partially neutralized to a pH of from about 5 to about 7 and/or comprise a bioabsorbable alpha-cyanoacrylate adhesive composition.

In another embodiment, the devices comprise a first and second barrier layer, a first and second adhesive layer, a middle barrier layer comprising a hole, and a carrier disk comprising a bioactive agent and disposed in the hole of the middle barrier layer. In this embodiment, the lower surface of the first barrier layer is laminated to the upper surface of the first adhesive layer, the lower surface of the first adhesive layer is laminated to the upper surface of the middle barrier layer, the lower surface of the middle barrier layer is laminated to the upper surface of the second adhesive layer, and the lower surface of the second adhesive layer is laminated to the upper surface of the second barrier layer.

In each embodiment described herein, the carrier and barrier layers comprise a biocompatible, biodegradable polymer.

These and other objects of the invention will be apparent from the following description and appended claims, and from practice of the invention.

### BRIEF DESCRIPTION OF THE FIGURES

FIG. 1 illustrates a side view of an embodiment of the device of the invention comprising a carrier layer and a first oxidized regenerated cellulose (ORC) layer.

FIG. 2 illustrates a side view of an embodiment of the device of the invention shown in FIG. 1 further comprising a second ORC layer laminated to the lower surface of the carrier layer.

FIG. 3a illustrates a side view of a partially encapsulated absorbable multilayer polymeric film of the invention comprising carrier layer, a first ORC layer, and a first and second barrier layer.

FIG. 3b illustrates a side view of another embodiment of the device of the invention comprising a first ORC layer laminated to the upper surface of a carrier layer and a second barrier layer laminated to the lower surface of the carrier layer.

FIG. 3c illustrates a side view of another embodiment of the device of the invention comprising a first ORC layer laminated to the upper surface of a first barrier layer and a carrier layer laminated to the lower surface of a first barrier layer 50.

FIG. 3d illustrates a side view of a sustained release device comprising a carrier layer laminated to the upper surface of a second barrier layer.

FIG. 3e illustrates a side view of another embodiment of the device of the invention wherein two adhesive polymeric layers are laminated between the carrier layer and the first and second barrier layers.

FIG. 4a illustrates a side view of the sustained release device shown in FIG. 3a further comprising a second ORC layer laminated to the lower surface of the second barrier layer.

FIG. 4b illustrates a side view of the embodiment shown in FIG. 4a further comprising two adhesive polymeric layers laminated between a carrier layer and first and second barrier layers.

FIG. 5a illustrates a side view of a fully encapsulated sustained release device of the invention comprising first and second barrier layers, a middle barrier layer comprising a hole, first and second adhesive layers, and a carrier disk comprising a bioactive agent(s) and disposed in the hole of the middle barrier layer.

FIG. 5b illustrates a perspective view of the middle barrier layer comprising a hole from the embodiment illustrated in FIG. 5a.

FIG. 6 illustrates an embodiment of the invention comprising a polymer carrier layer and an ORC layer, specifically an absorbable adhesion barrier sold under the trademark INTERCEED® (available from GYNECARE, a division of Ethicon, Inc., Somerville, N.J.).

FIG. 7 illustrates an embodiment of the invention comprising a polymer carrier layer and an ORC material, specifically an absorbable hemostat sold under the trademark SURGICEL® ORIGINAL™ (available from Johnson & Johnson Wound Management, a division of Ethicon, Inc., Somerville, N.J.).

FIG. 8 illustrates an embodiment of the invention comprising a polymer carrier layer and an ORC material, specifically an absorbable hemostat sold under the trademark SURGICEL® NU-KNIT® (available from Johnson & Johnson Wound Management, a division of Ethicon, Inc., Somerville, N.J.).

FIG. 9 illustrates a PCL/PGA 36/64 film prepared by compression molding.

FIG. 10 illustrates the cumulative release (percent) of ketoprofen (KTP) at 10 percent w/w, showing various early release profiles between day 0 and day 7.

FIG. 11 illustrates the cumulative release (percent) of KTP at 10 percent w/w, day 0-25.

FIG. 12 illustrates the normalized amount of KTP released (mg per day per 2 sq·cm) at 10 percent w/w, day 0-7.

FIG. 13 illustrates the amount of KTP released (mg per day per 2 sq. cm) of KTP at 10 percent w/w, day 0-25.

FIG. 14 illustrates the normalized release (percent) of KTP at 10 percent w/w, day 0-7.

FIG. 15 illustrates the cumulative release (percent) of KTP at 10 to 30 percent w/w (Monolayer vs. 250 um thick PCL/PGA polymeric barrier).

FIG. 16 illustrates the amount of KTP Released at 10 to 30 percent w/w (Monolayer vs. 250 um thick PCL/PGA polymeric barrier).

FIG. 17 illustrates a perforated ORC-polymeric film multilayer construct.

FIG. 18 illustrates a laminated ORC-polymeric strip multilayer film construct.

FIG. 19 illustrates an expandable weave of ORC-polymeric film multilayer construct.

FIG. 20 illustrates a porcine GI tract partially covered with 60 uL 2-Octyl Cyanoacrylate (2OCA).

FIG. 21 illustrates a porcine GI tract partially covered with an ORC-polymeric film impregnated with 60 uL 2OCA.

FIG. 22 illustrates the cumulative release of bimatoprost from films at 30 percent loading w/w.

FIG. 23 illustrates the amount of 5-FU released from films (mg per day per 2.5 mm disk) with 5-FU loading of 30 percent w/w, day 0-25.

FIG. 24 illustrates dispensing 2-Octyl Cyanoacrylate sealant onto an ORC layer of an ORC-PCL/PGA film.

FIG. 25 illustrates a colon anastomosis (suture) in rabbit.

FIG. 26 illustrates a rabbit colon repaired with an ORC-PCL/PGA film as an adjunct to suture.

FIG. 27 illustrates the cumulative release of infliximab from films.

### DETAILED DESCRIPTION OF THE INVENTION

The invention provides a customizable solvent-free sustained release device for use in internal wound closure, such as on the gastrointestinal tract, decreasing or modulating the local inflammation levels at a wound site in order to promote wound healing, and sustained release of a bioactive agent, such as an anti-inflammatory, pain killer, or chemotherapeutic agent, whereby the release profile of the device is customizable. As used herein, solvent free means that solvents are not used in the preparation of the sustained release device.

The device of the invention is composed of at least a polymeric carrier layer and optionally an oxidized regenerated cellulose layer(s). The polymeric carrier layers or films of the invention can be processed by cutting the films into various sizes and shapes, punching the films into disks, and/or conducting an additional lamination procedure on the films to obtain multi layer films as described in the examples below. It will be understood by persons skilled in the art that a mono layer film (carrier layer) can contain more than one drug.

Polymeric barrier layers can be added to modify the release profile (burst, time of release, etc.) of the selected bioactive agent(s), and adhesive layers can be added to allow for the formulation of a bioactive agent(s) that is heat sensitive. For instance, as will be described herein and in the Examples below, the burst at 24 hours can be customized between 0 and over 60 percent for a device or film loaded at 10 percent w/w in KTP. The burst at 24 hours can reach 80 percent for devices or films loaded at 30 percent w/w. Furthermore, true sustained release of ketoprofren (KTP) can be achieved over time when the carrier layer is laminated between two (2) or more layers of PCL/PGA of various thicknesses, e.g., typically between 25 and 500 um each.

The variety of biodegradable polymers, the sizes, the shapes, and the various constructions discussed herein allow for customized delivery of hydrophilic, hydrophobic, and heat sensitive compounds. Multilayer partially encapsulated polymeric films allow for bimodal release of actives, and multilayer fully encapsulated polymeric films allow for delayed sustained release of actives.

One embodiment of the invention is based on the unexpected finding that a selected construction (e.g. fully encapsulated sustained release device with PCL/PGA 36/64) has a tendency to lead to the same release profile (delayed sustained for 28-30 days) regardless of the hydrophilicity of the drug. For multilayer fully encapsulated films containing two adhesives layers between the carrier layer and the barrier layers, low lamination temperature (55 degrees Celsius) lead to zero order release profile of bimatoprost while higher lamination temperature (60 degrees Celsius) lead to a delayed sustained release of the bioactive agent, specifically bimatoprost.

The additional lamination of an oxidized regenerated cellulose (ORC) material (native or further engineered) onto a film allow for: adhesion of the polymeric film to specific organ(s) or area(s) of the body; local hemostasis; and increase of drug uptake by tumors.

Because of the high density achieved with the manufacturing approach disclosed herein, the drug loadings, while being drug dependent, can reach levels up to 70 percent w/w. In one embodiment, the drug loading is in the range of about 10 percent to about 30 percent by weight. Moreover, no preformulation is needed (solvent free). The inventions herein are advantageous with very hydrophobic compounds, compounds that are only stable in a lyophilized environment, and the simultaneous formulation of various compounds.

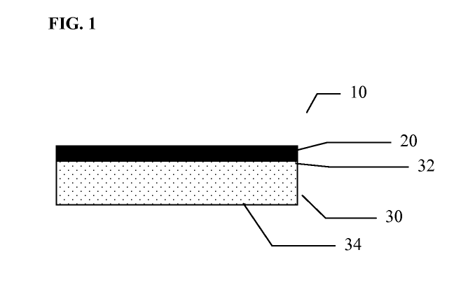

FIG. 1 illustrates one embodiment of a sustained release device 10 according to the invention comprising a carrier layer 30 and a first oxidized regenerated cellulose (ORC) layer 20. The carrier layer 30, having an upper surface 32 and a lower surface 34 defining a thickness of the carrier layer 30 there between, comprises a biocompatible, biodegradable polymer and bioactive agent. The first ORC layer 20 is laminated to the upper surface 32 of the carrier layer 30. The release profile of this embodiment is a burst effect in which a large drug amount is quickly released into the body. The size of the burst and the length of the burst that typically varies between 0.3 to 3 days are a function of the nature of the polymer used, the drug load, the geometry of the film (thickness, aspect ratio) and the presence or absence of plasticizers or porogens. Since the drug release is mainly limited by diffusion, the burst will increase as the hydrophilicity of the polymer increases, the film thickness decreases, and the surface-to-volume ratio increases. The presence of plasticizers or porogens will also increase the burst as they are displaced into water-based solutions or bodily fluids, allowing for the bioactive agent(s) to be released faster in the medium.

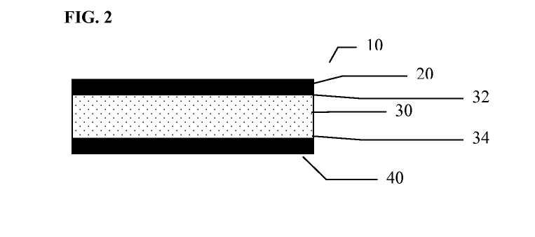

In another embodiment of the invention, the device can comprise a second ORC layer 40 laminated to the lower surface 34 of the carrier layer 30, as illustrated in FIG. 2. The release profile of this embodiment is a burst like the embodiment shown in FIG. 1, but additional treatment of one of the ORC layers (e.g. addition of cyanoacrylate sealant to a dense and thick ORC layer such as that sold under the trademark SURGICEL® NU-KNIT® Absorbable Hemostat (available from Johnson & Johnson Wound Management, a division of Ethicon, Inc., Somerville, N.J.)) may lead to unidirectional release of the selected active(s) while native ORC layers will lead to bidirectional release of the selected active(s). As used herein, the term “native ORC layer” refers to an ORC layer that has not been combined with an internal sealant, such as a cyanoacrylate.

For instance, if a sealant such as a cyanoacrylate is added to a thick (greater than 0.5 mm) ORC fabric (such as the absorbable hemostat sold under the trademark SURGICEL® NU-KNIT® (available from Johnson & Johnson Wound Management, a division of Ethicon, Inc., Somerville, N.J.)) in amounts that allow for saturation of the said ORC, the ORC/cyanoacrylate construct may effectively act as a barrier resulting in a unidirectional or preferential release of the active toward the opposite side of the construct. Conversely, if the ORC layer(s) is (are) not combined with any type of sealant or if a sealant is used in limited amount and onto an open ORC fabric (such as that sold under the trademark SURGICEL® ORIGINAL™ (available from Johnson & Johnson Wound Management, a division of Ethicon, Inc., Somerville, N.J.)), then one can expect a bidirectional release of the selected active(s).

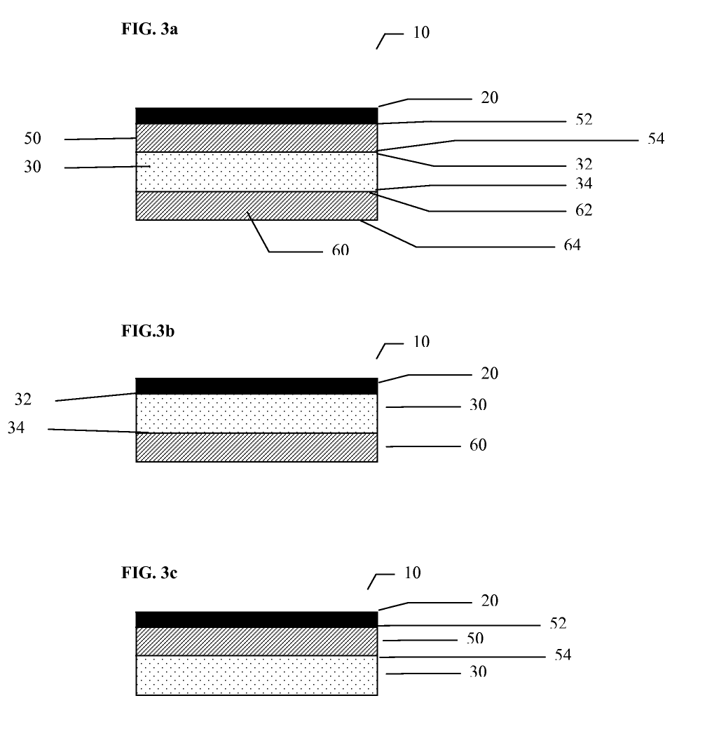

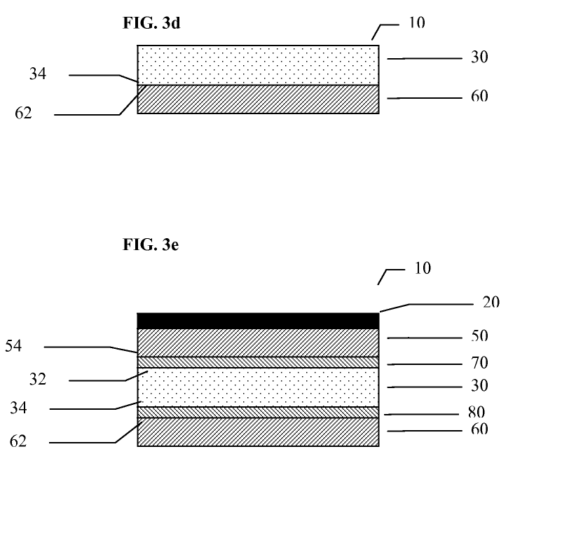

FIG. 3a illustrates another embodiment, namely a partially encapsulated absorbable multilayer polymeric film. The sustained release device 10 according to the invention shown in FIG. 3a comprises a carrier layer 30, a first ORC layer 20, and a first 50 and second 60 barrier layer. The carrier layer 30, having an upper surface 32 and a lower surface 34, comprises a biocompatible, biodegradable polymer and bioactive agent. The first barrier layer 50, having an upper surface 52 and a lower surface 54, comprises a biocompatible, biodegradable polymer, and the lower surface 54 of the first barrier layer 50 is laminated to the upper surface 32 of the carrier layer 30. The second barrier layer 60, having an upper surface 62 and a lower surface 64, comprises a biocompatible, biodegradable polymer, and the upper surface 62 of the second barrier layer 60 is laminated to the lower surface 34 of the carrier layer 30. The first ORC layer 20 is laminated to the upper surface 52 of the first barrier layer 50.

The release profile for the embodiment illustrated in FIG. 3a is a bimodal release profile since an initial burst is observed as the partially encapsulated bioactive agent (i.e., the carrier layer 30 comprising the bioactive agent is encapsulated between two barrier layers 50 and 60) is released from the open sides at the periphery of the carrier layer 30. This is followed by a period of no or undetectable release of approximately 10 days then by a delayed sustained release of the bioactive agent(s) that diffuses through the first 50 and second 60 barrier layers and ORC layer 20 of the device or film.

In another embodiment of the invention, two adhesive polymeric layers (made of PCL/PGA X/100-X, where X varies from 100 to 70 in one embodiment and from 95 to 80 in another embodiment) can be laminated between the carrier layer 30 and the first and second barrier layers 50 and 60 (FIG. 3e). Specifically, as shown in FIG. 3e, a first adhesive layer 70 is laminated on the upper surface 32 of the carrier layer 30 and bottom surface 52 of the first barrier layer 50, and a second adhesive layer 80 is laminated on the lower surface 34 of the carrier layer 30 and the upper surface 62 of the second barrier layer 60. The adhesive layers 70 and 80 have a low melting point and do not have any impact on the release profile of the device. While the release profile of this embodiment is similar to the one described above, this approach allows for the formulation of a bioactive agent(s) that is heat sensitive, or a bioactive agent that degrades or decomposes below the processing temperature of PCL-PGA 36/64, 120 degrees Celsius. For example, bimatoprost decomposes at 70 degrees Celsius.

Multilayer sustained release constructions comprising various layers laminated together is also possible. Specifically, in one embodiment, shown in FIG. 3b, a multilayer sustained release device according to the invention comprises the first ORC layer 20 laminated to the upper surface 32 of a carrier layer 30 and the second barrier layer 60 laminated to the lower surface 34 of the carrier layer 30. In another embodiment, shown in FIG. 3c, the first ORC layer 20 is laminated to the upper surface 52 of the first barrier layer 50 and the carrier layer 30 is laminated to the lower surface 54 of the first barrier layer 50. In yet another embodiment of the invention, the sustained release device 10 comprises only a first or second barrier 50 and 60, not both as illustrated in FIG. 3d. In FIG. 3d, the multilayer sustained release device comprises the lower surface 34 of the carrier layer 30 laminated to the upper surface 62 of the second barrier layer 60.

The release profile for the embodiments shown in FIGS. 3b, 3c, and 3d are expected to be the same if native ORC material (not combined with an internal sealant as discussed in more detail below) is used. The release profile for these embodiments is expected to be a burst or a slow release depending on the solubility of the bioactive agent(s). It is expected that the bioactive agent will be released from the open sides at the periphery of the carrier layer 30 and through the ORC layer (FIGS. 3b and 3c only), which will not impact the release profile if not combined with an internal sealant. Thus, the initial burst as well as the delayed sustained release would be slower than the release profile of the embodiment shown in FIG. 2. If the bioactive agent(s) is soluble, the release profile of the embodiments shown in FIGS. 3b, 3c, and 3d will likely be closer to that of the embodiment shown in FIG. 2.

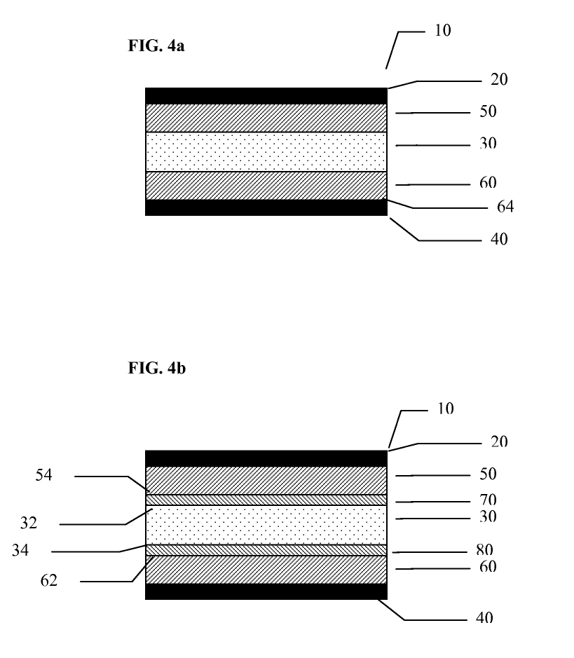

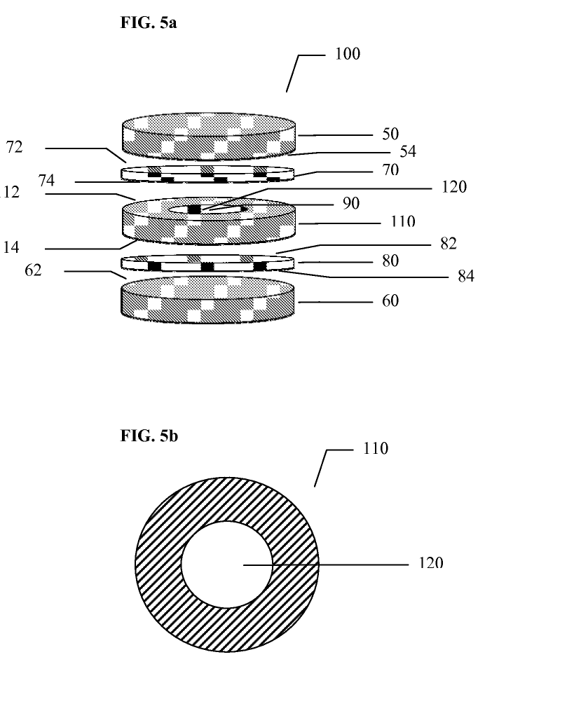

In another embodiment of the invention, the sustained release device 10 shown in FIG. 3a comprises a second ORC layer 40 laminated to the lower surface 64 of the second barrier layer 60 as illustrated in FIG. 4a. The release profile of this embodiment is a burst like the embodiment shown in FIG. 3a, but, as discussed above, additional treatment of one of the ORC layers (e.g. addition of cyanoacrylate sealant to a dense and thick ORC layer such as SURGICEL® NU-KNIT® Absorbable Hemostat (available from Johnson & Johnson Wound Management, a division of Ethicon, Inc., Somerville, N.J.)) may lead to unidirectional release of the selected active(s) while native ORC layers will lead to bidirectional release of the selected active(s).

FIG. 4b illustrates the embodiment of FIG. 4a further comprising two adhesive polymeric layers (made of PCL/PGA X/100-X, where X varies from 100 to 70 in one embodiment and from 95 to 80 in another embodiment) laminated between the carrier layer 30 and the first and second barrier layers 50 and 60. Specifically, as shown in FIG. 4b, a first adhesive layer 70 is laminated on the upper surface 32 of the carrier layer 30 and bottom surface 54 of the first barrier layer 50, and a second adhesive layer 80 is laminated on the lower surface 34 of the carrier layer 30 and the upper surface 62 of the second barrier layer 60. While the release profile of this embodiment is similar to the one described above for FIG. 4a, this approach allows for the formulation of a bioactive agent(s) that is heat sensitive, or a bioactive agent that degrades or decomposes below the processing temperature of PCL-PGA 36/64, 120 degrees Celsius. For example, bimatoprost decomposes at 70 degrees Celsius.

FIG. 5a illustrates another embodiment of the invention, namely, a fully encapsulated sustained release device 100 according to the invention, comprising first 50 and second 60 barrier layers, a middle barrier layer 110 comprising a hole 120, first 70 and second 80 adhesive layers, and a carrier disk 90 comprising a bioactive agent(s) and disposed in the hole 120 of the middle barrier layer 110. FIG. 5b illustrates a perspective view of the middle barrier layer 110 comprising a hole 120. The barrier layers 50 and 60 in the embodiment illustrated in FIG. 5a can optionally be coated with ORC.

The first 50 and second 60 barrier layers, the first 70 and second 80 adhesive layers, and the middle barrier layer 110 each have an upper and a lower surface. The lower surface 54 of the first barrier layer 50 is laminated to the upper surface 72 of the first adhesive layer 70, the lower surface 74 of the first adhesive layer 70 is laminated to the upper surface 112 of the middle barrier layer 110, the lower surface 114 of the middle barrier layer 110 is laminated to the upper surface 82 of the second adhesive layer 80, and the lower surface 84 of the second adhesive layer 80 is laminated to the upper surface 62 of the second barrier layer 60. The first and second barrier layers 50 and 60, the first and second adhesive layers 70 and 80, the middle barrier layer 110, and the carrier disk 90 each comprise a biocompatible, biodegradable polymer.

As demonstrated in Example 7 below, a fully encapsulated sustained release device 100 as shown in FIG. 5a (with PCL/PGA 36/64) has a tendency to lead to the similar release profile (delayed sustained for 28-30 days) regardless of the hydrophilicity of the drug. For this embodiment containing two adhesive layers between the carrier layer 30 and the barrier layers 50 and 60, low lamination temperature (about 55 degrees Celsius or lower) lead to zero order release profile of bimatoprost while higher lamination temperature (about 60 degrees Celsius or higher) lead to a delayed sustained release of the active.

The carrier layer 30 and carrier disk 90 comprise a biocompatible, biodegradable polymer and a bioactive agent(s) to be released under a selected profile. The biodegradable polymers readily break down into small segments when exposed to moist body tissue. The segments then are either absorbed by or passed from the body. More particularly, the biodegraded segments do not elicit permanent chronic foreign body reaction, because they are absorbed by the body or passed from the body such that no permanent trace or residual of the segment is retained by the body. For the purposes of this invention the terms bioabsorbable and biodegradable are used interchangeably.

The biocompatible, biodegradable polymers may be natural, modified natural, or synthetic biodegradable polymers, including homopolymers, copolymers, and block polymers, linear or branched, segmented or random, as well as combinations thereof. Particularly well suited synthetic biodegradable polymers are aliphatic polyesters which include but are not limited to homopolymers and copolymers of lactide (which includes D(−)-lactic acid, L(+)-lactic acid, L(−)-lactide, D(+)-lactide, and meso-lactide), glycolide (including glycolic acid), epsilon-caprolactone, p-dioxanone (1,4-dioxan-2-one), and trimethylene carbonate (1,3-dioxan-2-one). Other suitable polymers are polycaprolactone (PCL), poly(lactic-co-glycolic acid) (PLGA), or poly(glycolide-co-caprolactone) (PCL/PGA). Within the PCL/PGA family of polymers, the preferred polymers are 36/64 and 90/10. Other biodegradable polymers are also suitable and would be known to those skilled in the art.

The bioactive agent(s) to be released from the carrier layer 30 and carrier disk 90 can be an anti-inflammatory or a pain killer such as ketoprofen (KTP), or an antibacterial such as gentamicin. Other actives such as chemotherapeutic agents can be used as well such as 5-FU, paclitaxel, and the anti-cancer pharmaceutical preparation TAXOTERE® (marketed by Aventis Pharma S.A.). In addition, the release of bimatoprost (used to control the progression of glaucoma and in the management of ocular hypertension) has been evaluated.

The variety of bioactive agents that can be used in conjunction with the polymers of the invention is vast. In general, bioactive agents which may be administered via pharmaceutical compositions of the invention include, without limitation, antiinfectives, such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators, including general coronary, peripheral and cerebral; central nervous system stimulants; cough and cold preparations, including decongestants; hormones, such as estradiol and other steroids, including corticosteroids; hypnotics; immunosuppressives; muscle relaxants; parasympatholytics; psychostimulants; sedatives; tranquilizers; naturally derived or genetically engineered proteins, growth factors, polysaccharides, glycoproteins or lipoproteins; oligonucleotides; antibodies; antigens; cholinergics; chemotherapeutics; hemostatics; clot dissolving agents; radioactive agents; and cystostatics.

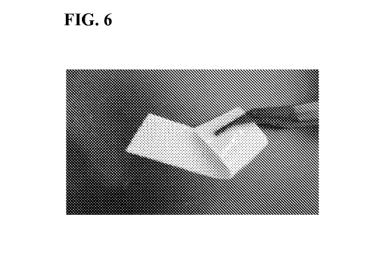

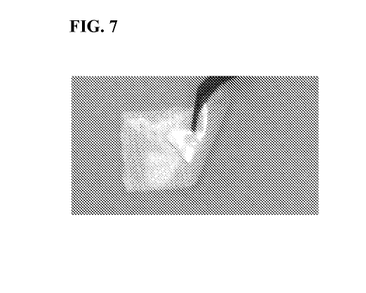

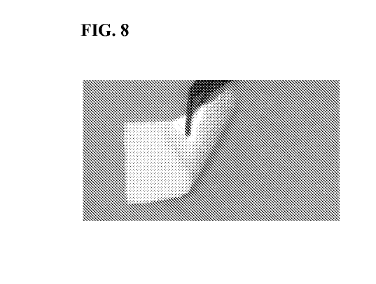

The ORC of the first and second ORC layers 20 and 40 are provided in various forms such as woven fabrics, nonwoven fabrics, foams, particles, and the like. Suitable ORC fabrics include absorbable hemostats and absorbable adhesion prevention barriers. Several constructs comprising a PCL/PGA 36/64 polymer carrier layer and an ORC layer 20 were manufactured with selected ORC fabrics. In one embodiment, the ORC is a woven fabric, such as an absorbable adhesion barrier sold under the trademark INTERCEED® (available from GYNECARE, a division of Ethicon, Inc., Somerville, N.J.), an absorbable hemostat sold under the trademark SURGICEL® ORIGINAL™ (available from Johnson & Johnson Wound Management, a division of Ethicon, Inc., Somerville, N.J.), or an absorbable hemostat sold under the trademark SURGICEL® NU-KNIT® (available from Johnson & Johnson Wound Management, a division of Ethicon, Inc., Somerville, N.J.), as shown in FIGS. 6, 7, and 8, respectively. In another embodiment, the ORC is a nonwoven fabric such as ORC absorbable hemostat sold under the trademark SURGICEL® FIBRILLAR™ (available from Johnson & Johnson Wound Management, a division of Ethicon, Inc., Somerville, N.J.). The foregoing examples of suitable ORC materials are considered native ORC material.

Laminating an ORC layer (native or further engineered) onto a device or film of the inventions has several advantages. First, the ORC layer can be laminated onto a carrier or barrier layer to serve as a local hemostat, such as when native ORC material such as an absorbable hemostat sold under the trademark SURGICEL® ORIGINAL™, an absorbable hemostat sold under the trademark SURGICEL® FIBRILLAR™, or an absorbable hemostat sold under the trademark SURGICEL® NU-KNIT® are utilized. Second, the ORC layer can be laminated onto a carrier or barrier layer to provide hemostasis or to prevent adhesions, such as when an absorbable adhesion barrier sold under the trademark INTERCEED® is utilized. Third, the ORC layer can be further oxidized to lower the pH and enhance the uptake of a bioactive agent(s). For instance, it has been demonstrated that the uptake of 5-FU by tumors can be augmented when the local pH in the vicinity of the tumor cells is diminished as compared to the pH inside of the cells (see A S E Ojugo et al. Brit, J. Cancer 77(6): 873-879, 1998).

Fourth, the ORC layer can be partially neutralized so as to combine with an internal sealant such as a cyanoacrylate. Specifically, the first and/or second ORC layer 20 and/or 40 can be coated with a sealant either at the time of manufacturing or at the time of surgery. While different types of sealants (synthetic or biologic) can be used, synthetic sealants such as cyanoacrylates (e.g., 2-Octyl Cyanoacrylate or Butyl Cyanoacrylate) have been evaluated. As discussed in U.S. patent application Ser. No. 12/645,164, published on Jun. 23, 2011, as U.S. Patent Publication No. 2011/0152924 A1, incorporated herein by reference, the ORC layer(s) has to undergo a pre-treatment (partial neutralization to a pH of from about 3.5 to about 7 followed by lyophilization) to make it compatible with cyanoacrylate sealants. In one embodiment, the ORC layer(s) has to be partially neutralized to a pH from about 4.5 to about 6.5 followed by lyophilization. In summary, the ORC fabric is partially neutralized in a 1 percent w/w sodium bicarbonate solution for typically 30 to 200 seconds (preferably 90 to 120 seconds) then padded dry prior to being lyophilized and stored under dry conditions.

The release of the bioactive agent(s) is guided by diffusion from the carrier layer and through any barrier, adhesive, or ORC layers. Native ORC material, or ORC material that has not been combined with an internal sealant such as a cyanoacrylate, will not impact the release profile of a bioactive agent(s). An ORC material that has been combined with an internal sealant such as a cyanoacrylate, however, is expected to act like a barrier and decrease the release rate of a bioactive agent(s).

The first, second, and middle barrier layers, 50, 60, and 110, respectively, comprise a single layer of a biocompatible, biodegradable polymer. In another embodiment, the first, second, and middle barrier layers, 50, 60, and 110, respectively, can be a laminate of several layers of polymer. The polymeric barrier layers can be prepared as a pure polymer or as mixture of polymer. Furthermore, this polymer can, but does not have to be similar to the one used in the carrier. In yet another embodiment, the barrier layers 50 and 60 and middle barrier layer 110 can be made either of a selected polymer or of a polymer supplemented with a plasticizer. Specifically, the barrier layer can also contain one or more plasticizers (hydrophilic such as tri-ethyl citrate (TEC) or hydrophobic such as tri-butyl acetyl citrate (TBAC)) and/or active(s), or a combination thereof, such as 30 percent TEC and 12 percent TBAC as described in Table 3 of Example 2. When the addition of a plasticizer is desired, it is admixed to the polymer, and then the mixture is poured into a suitable stainless steel mold.

Suitable polymers for the first and second barrier layers 50 and 60 and the middle barrier layer 110 are the same as those suitable for the carrier layer 30 or carrier disk 90 discussed above. While the polymer(s) of the barrier layer(s) can be similar to the polymer(s) used in the carrier layer 30 or carrier disk 90, it is not necessary. See Examples 1 and 2 below.

The barrier layers control or modulate the diffusion, therefore the release, of the bioactive agent(s) from the carrier layer. Therefore, to further customize the release of the bioactive agent(s), it is in the scope of this invention to consider the barrier layers, specifically the first 50 and second 60 barrier layers described in FIGS. 3, 4 and 5, to be monolayers or to be multilayer (the result of a multilayer lamination process). In either case, any of the layers or the sub-layers can be made of the same or different polymer. These layers can also contain a variety of moieties such as plasticizers, porogens, or stabilizing agents.

Plasticizers incorporated in any of the barrier and or carrier layers can either be hydrophilic, such as tri-ethyl citrate (TEC), or hydrophobic, such as tri-butyl acetyl citrate (TBAC). Polysorbates, tweens, porogens, and stabilizing agents (e.g. trehalose, zinc sulfate, and amino acids) can also be used as plasticizers. The plasticizer can be chosen so that its degree of hydrophilicity is such that it acts as a dispersant for the active to be released. It can also be chosen to protect the active from the local environment (e.g. bodily fluid) by complexation or ionic bonding or hydrophobic bonding (e.g. with peptides or proteins). When the addition of a plasticizer is desired for a barrier layer, it is admixed to the polymer, and then the mixture is poured into a suitable stainless steel mold.

The adhesive layers 70 and 80 comprise PCL/PGA X/100-X, where X varies from 100 to 70 in one embodiment and from 95 to 80 in another embodiment. In one embodiment, one or both adhesive layers 70 and 80 comprise a random copolymer of PCL/PGA 90/10. The adhesive layers 70 and 80 have a low melting point and do not have any impact on the release profile of the device. The lower melting point of the adhesive layers 70 and 80 comes from the low glass transition temperature combined with a drop in crystallinity.

With respect to the embodiments illustrated in FIGS. 1-4, the carrier layer 30, generally having an upper surface 32 and a lower surface 34, is prepared by admixing the polymer(s) with the bioactive agent(s) in selected ratios followed by lamination under high pressure (compression molding) in a stainless steel mold under solvent-free conditions, such as set forth in Example 1. The first ORC layer 20 is laminated onto the upper surface 32 of the carrier layer 30 by compression molding such that part of the ORC fibers of the ORC layer 20 are embedded into the carrier layer 30, and part of the ORC fibers of the ORC layer 20 remain exposed. See Example 4. The exposed ORC fibers of the ORC layer can be used to glue the ORC layer 20 to a selected tissue at the time of surgery following the addition of a sealant, as discussed above. The exposed ORC fibers of the ORC layer 20 can also be used as a hemostat and/or as a means to decrease the local pH in order to enhance the drug uptake by an organ or tumor. The second ORC layer 40 can be laminated onto the lower surface 34 of the carrier layer 30 using the same compression molding method discussed above with respect to the first ORC layer 20.

The ORC layer(s) 20 and/or 40 are laminated onto the carrier layer 30 or the barrier layer(s) 50 and/or 60 by compression molding under moderate temperature. The lamination temperature is a function of the melting point of the polymeric carrier layer 30 or barrier 50 and/or 60 layer(s). The pressure is typically very low, such as 5-50 psi, as compared to the pressure required to glue two polymeric layers.

ORC-polymeric film constructs, such as those prepared as described in Example 4, can be further engineered during or after the final lamination step based on the mechanical properties it has to display. For instance, for enhanced stiffness, the ORC-polymeric film can be manufactured into a laminated sheet, such as that shown in FIGS. 6, 7, and 8, by compression molding an ORC fabric with PCL/PGA 36/64 at 125 degrees Celsius and 25,000 lbs of pressure for 8 minutes. The polymeric film acts as a buttress to the ORC fabric.

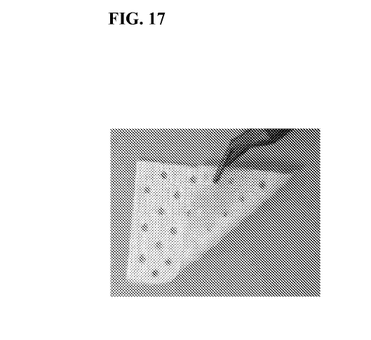

Alternatively, an ORC-polymeric film can be perforated, as shown in FIG. 17, to further customize the release profile of selected active(s) or to allow for a sealant or glue such as cyanoacrylate sealant to be added at the time of surgery. The ORC-polymeric film is perforated by punching (die), and the perforation modifies the release profile of the film by acting as a partially encapsulated disk.

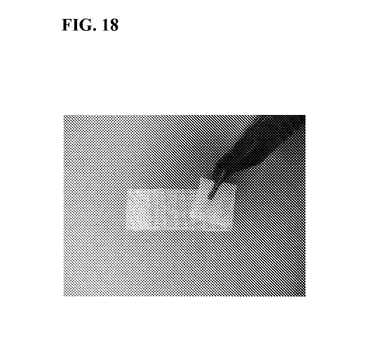

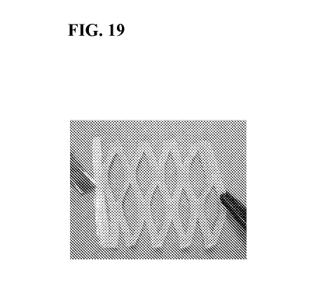

In addition, if flexibility and elongation are required, an ORC-polymeric film can be made of laminated strips (FIG. 18) or expandable “weaves” (FIG. 19). Once the polymeric film is made, it is cut into strips and the ORC fabric is laminated onto the strips at 125 degrees Celsius and 30-100 lbs of pressure in order to form the laminated strips. The expandable “weaves” can be made by making a series of parallel cuts approximately ½ inch long and ⅛ inch apart.

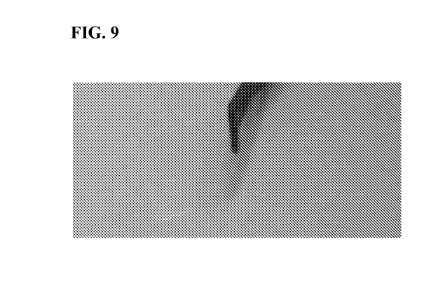

The thickness of the barrier layer is customizable and typically varies between 25 and 250 um. See Example 2 where several versions of a barrier layer were prepared. FIG. 9 shows a PCL/PGA 36/64 film prepared by compression molding.

The thickness of the carrier layer typically varies between 25 and 5000 um. In one embodiment, the thickness of the carrier layer is between 100 and 1000 um. In addition, several carrier layers can be laminated onto each other. The thickness of the adhesive layer typically varies between 1 and 1000 um. In one embodiment, the thickness of the adhesive layer is between 10 and 100 um.

The diameter of the carrier disk, which may be formed from a carrier layer, typically varies between 0.25 and 20 mm. In one embodiment, the thickness of the carrier disk is between 1 and 5 mm.

The sustained release device may be of any suitable shape. Suitable shapes include, but are not limited to circle, square, rectangle, triangle, cylinder, cube, and the like. One skilled in the art would understand how to modify the shape and size, including the length, of the devices of the invention based on one's anticipated outcome, including but not limited to, intended use of the device and intended dosage and release profile of a bioactive agent(s).

Like the carrier layer 30, the first and second barrier layers 50 and 60 each have an upper surface and a lower surface, and the lower surface 54 of the first barrier layer 50 is laminated onto the upper surface 32 of the carrier layer 30 by compression molding as set forth in Example 2 and shown in FIG. 3a. The upper surface 62 of the second barrier layer 60 is also laminated onto the lower surface 34 of the carrier layer 30 by compression molding. The first ORC 20 layer is laminated onto the upper surface 52 of the first barrier layer 50 by compression molding the same way the first ORC layer 20 can be laminated onto the carrier layer 30, as set forth above. The second ORC layer 40 (FIG. 4a) can be laminated onto the lower surface 64 of the second barrier layer 60 using the same compression molding method discussed above with respect to the first ORC layer 20.

Example 6 below explains in detail how the embodiment illustrated in FIG. 5a is formulated and prepared. Generally, however, the layers of the embodiment in FIG. 5a are made individually by compression molding under heat and solvent-free conditions, and the layers are laminated together by compression molding under heat and solvent-free conditions. In one embodiment, the carrier layer from which the carrier disk 90 is formed comprises PCL/PGA 36/64, PCL/PGA 90/10, or PCL plus, optionally, plasticizer(s) and/or stabilizing agents. Suitable stabilizing agents are a function of the bioactive agent(s) to be released and include zinc sulphate, calcium chloride, arginine, methionine, and histidine. The first and second barrier layers 50 and 60 comprise PCL/PGA 36/64, plus, optionally, plasticizer(s) and/or stabilizing agents. The first and second adhesive layers 70 and 80 comprise PCL/PGA 90/10 or PCL.

The following examples are illustrative of the principles and practice of this invention, although not limited thereto. Numerous additional embodiments within the scope and spirit of the invention will become apparent to those skilled in the art once having the benefit of this disclosure.

### EXAMPLES

### Example 1

### Preparation of Monolayer Films Containing a Water Insoluble Drug (Ketoprofen)

This example describes the method of making of a monolayer polymeric drug loaded film by compression molding. The films, or carrier layers, prepared by this technique can be used as is or can be further processed as described in the additional examples below.

A drug relatively insoluble in water, such as ketoprofen, was ground into a fine powder in a mortar and admixed to a polymer powder (carrier) such as PCL/PGA (36/64) and placed into a stainless steel mold of suitable dimensions such, as 10 cm long×10 cm wide×250 um thick. Various drug loadings ranging from 10 to 30 percent w/w were evaluated for ketoprofen as described in Table 1. These ranges are not limited to 30 percent w/w and can further be increased.

The loaded molds were introduced into a press where compression combined with heating was applied under controlled conditions as described in Table 2.

TABLE 1
Ketoprofen loadings in PCL/PGA polymer

| | | | Keprofen load, |
|---|---|---|---|
| Sample | PCL/PGA, g | Ketoprofen, g | percent |
| 1 | 3.41960 | 0.37978 | 10 |
| 2 | 3.50000 | 0.70000 | 17 |
| 3 | 3.30000 | 0.82500 | 20 |
| 4 | 2.88930 | 1.23713 | 30 |

TABLE 2
Compression molding conditions for preparing Ketoprofen
monolayer films.

| | | Temperature, | |
|---|---|---|---|
| Step | Time, min | degrees Fahrenheit | Pressure, Lbs |
| 1 | 3 | 248 | 0 |
| 2 | 3 | 248 | 1,000 |
| 3 | 5 | 248 | 25,000 |
| 4 | 30 | 70 | 25,000 |
| 5 | 1 | 70 | 0 |

Upon completion of the heat/pressure cycle, the Ketoprofen-PCL/PGA films were removed from their molds and subsequently stored under vacuum or nitrogen atmosphere until use or further processing.

### Example 2

### Preparation of Multilayer Films Containing a Water Insoluble Drug (Ketoprofen)

This example describes the method of making of a multilayer polymeric drug loaded film by compression molding. The films prepared by this technique can be used as is or can be further processed, for example, by cutting to desired sizes or shapes, by punching, or by laminating additional polymeric layers.

The first step of this process was to prepare a monolayer film containing the drug of choice, such as ketoprofen, as described in Example 1.

The second step was the preparation of secondary polymeric film to be used as a polymeric barrier layer to modify the release profile of the selected drug. In two (2) of the samples prepared, a plasticizer was admixed to the polymer as described in Table 3. The polymer/plasticizer mixture was then poured into a suitable stainless steel mold.

TABLE 3
Compositions and contents of the barrier layers from 10 × 10 cm molds

| | PCL/PGA, | Plasticizer | Plasticizer | Thickness, |
|---|---|---|---|---|
| Sample | initial weight, g | initial weight, g | in-film, percent | um |
| 1 | 3.68940 | — | 0 | 250 |
| 2 | 3.0 | — | 0 | 125 |
| 3 | 2.0713 | 0.8847 | 30 | 125 |
| 4 | 2.0784 | 0.8955 | 12 | 125 |

The loaded molds were introduced into a press where compression combined with heating was applied under controlled conditions as described in Table 4.

TABLE 4
Compression molding conditions for preparing polymeric barrier layers.

| | | Temperature, | |
|---|---|---|---|
| Step | Time, min | degrees Fahrenheit | Pressure, lbs |
| 1 | 3 | 266 | 0 |
| 2 | 3 | 266 | 1,000 |
| 3 | 5 | 266 | 25,000 |
| 4 | 30 | 70 | 25,000 |
| 5 | 1 | 70 | 0 |

Upon completion of the heat/pressure cycle, the polymeric barrier layers were removed from their mold and subsequently stored under vacuum or nitrogen atmosphere until use or further processing. Once barrier layers of suitable size and shape were prepared, they were laminated on one or both sides (such as shown in FIG. 3a, but without the ORC layer 20, or FIG. 3d) of the polymeric drug carrier layers (from Example 1) to form multilayer films following a compression molding process similar to the one described above, but typically at lower lamination temperatures and pressures as detailed in Table 5. While not done in this experiment, one having ordinary skill in the art will appreciate that additional barrier layers can also be laminated onto existing barrier layers by following the same procedure.

TABLE 5
Compression molding conditions for laminating barrier layers onto carrier
layers or barrier layers added previously.

| | | Temperature, | |
|---|---|---|---|
| Step | Time, min | degrees Fahrenheit | Pressure, lbs |
| 1 | 8 | 213 | 180 |
| 2 | 15 | 70 | 180 |
| 3 | 1 | 70 | 0 |

### Example 3

### In-Vitro Release Studies of Ketoprofen from Monolayer and Multilayer Polymeric Films

In-Vitro release of ketoprofen (KTP) was quantified over time from 5 different film constructions (see below where X percent is the concentration w/w of KTP) and for 4 selected KTP concentrations (where X=10, 17, 20 and 30 percent w/w). The test articles, 2 cm long×1 cm wide, were immersed in a phosphate buffered saline (PBS) solution supplemented by 2 percent fetal bovine serum (FBS) onto a shaker in an incubator at 37 degrees Celsius. Release of KTP was quantified by HPLC-UV as follows: after filtration, aliquots were injected onto an ACE3-C18 column (50×4.6 mm×3 u with guard column). The KTP detection was performed via a UV Detector (Wavelength: 299 nm). The flow rate was 1.0 mL/min, and the mobile phase was a mixture of trifluoroacetic acid (TFA) (0.1 percent) and Acetonitrile. The tests articles consisted of a carrier layer made of PCL/PGA (36/64)+KTP (X percent). Four test articles included a barrier layer containing at least PCL/PGA (36/64) as described in Table 6.

TABLE 6
Loadings and construction characteristics of ketoprofen-based polymeric films

| | | | | Number of | |
|---|---|---|---|---|---|
| Film | | Carrier Layer | | Barrier Layers | |
| Construct | KTP loading, | Thickness, | Barrier Layer | per side of the | Barrier Layer |
| No. | percent | um | Thickness, um | Carrier Layer | Composition |
| 1 | X | 250 | 0 | 0 | N/A |
| | (Monolayer film, | | | | |
| | [FIG. 1 w/o ORC | | | | |
| | Layer]) | | | | |
| 2 | X | 250 | 125 | 1 | PCL/PGA 36/64 + |
| | (Multilayer film, | | | | 30 percent TEC |
| | [FIG. 3a w/o | | | | (Tri-ethyl Citrate) |
| | ORC Layer]) | | | | |
| 3 | X | 250 | 125 | 1 | PCL/PGA 36/64 + |
| | (Multilayer film, | | | | 11 percent TBAC |
| | [FIG. 3a w/o | | | | (Tri-butyl Acetyl |
| | ORC Layer]) | | | | Citrate) |
| 4 | X | 250 | 125 | 1 | PCL/PGA 36/64 |
| | (Multilayer film, | | | | |
| | [FIG. 3a w/o | | | | |
| | ORC Layer]) | | | | |
| 5 | X | 250 | 250 | 1 of 250 um | PCL/PGA 36/64 |
| | (Multilayer | | | Or | |
| | film), [FIG. 3a | | | 2 of 125 um | |
| | w/o ORC | | | each | |
| | Layer]) | | | | |

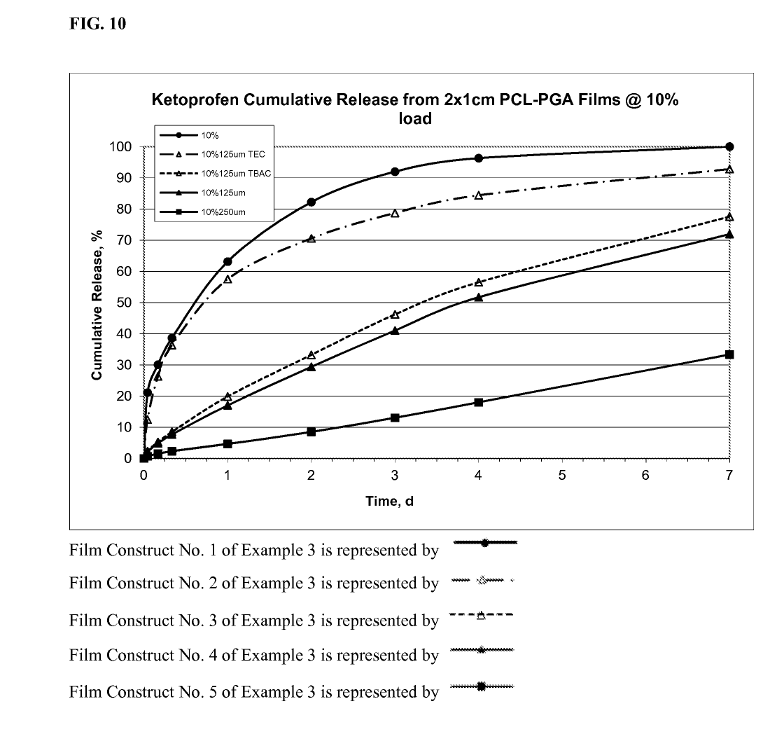

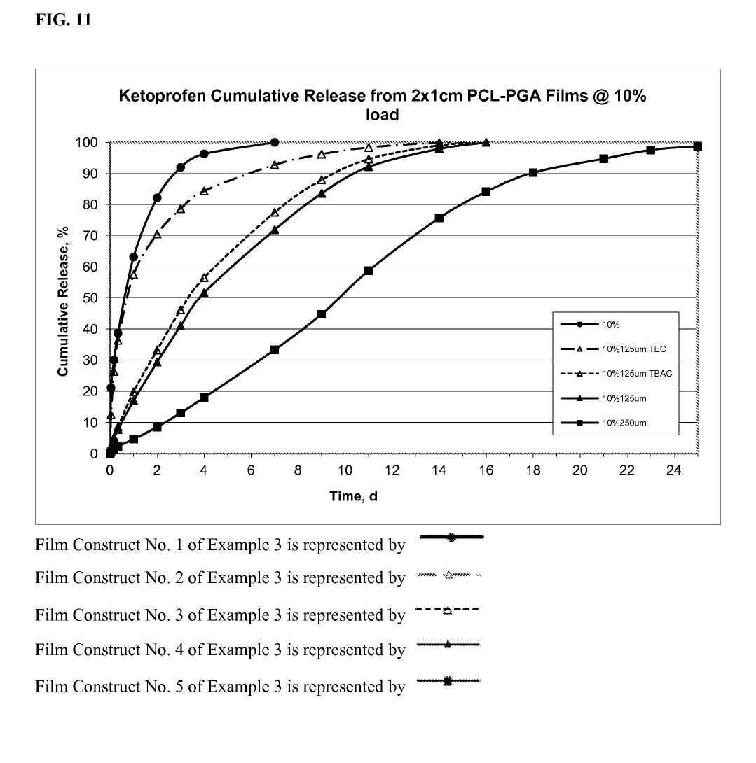

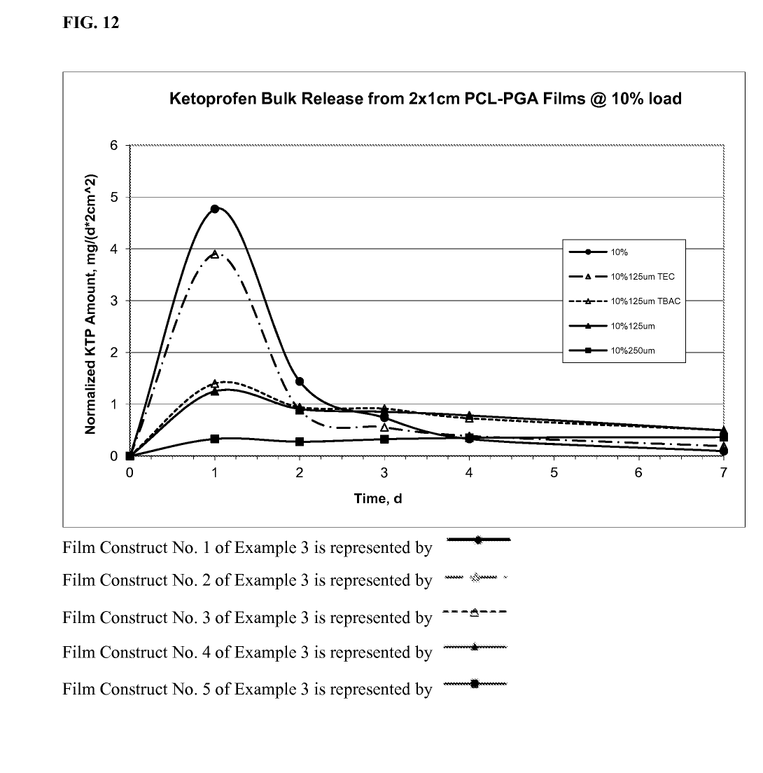

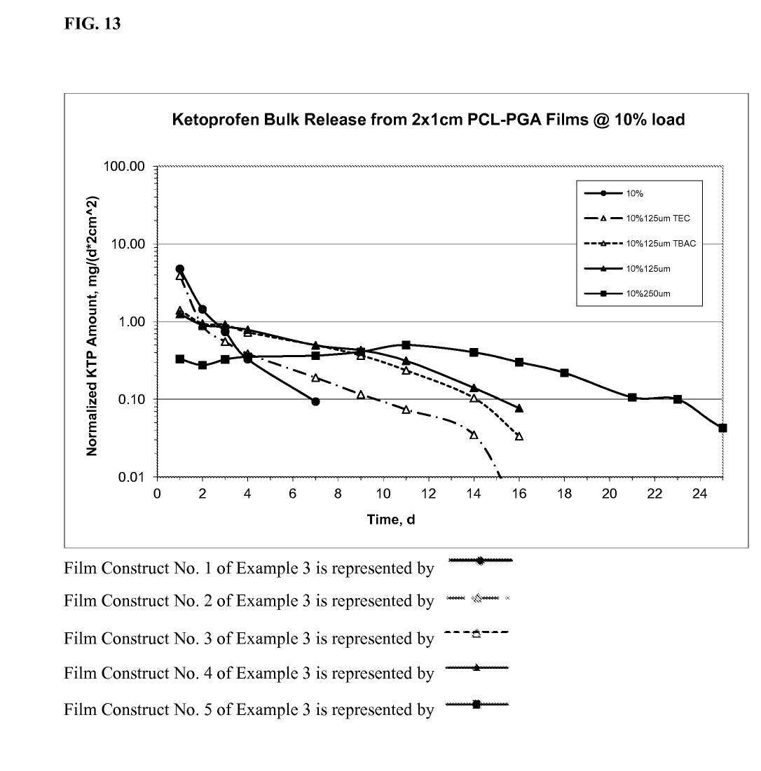

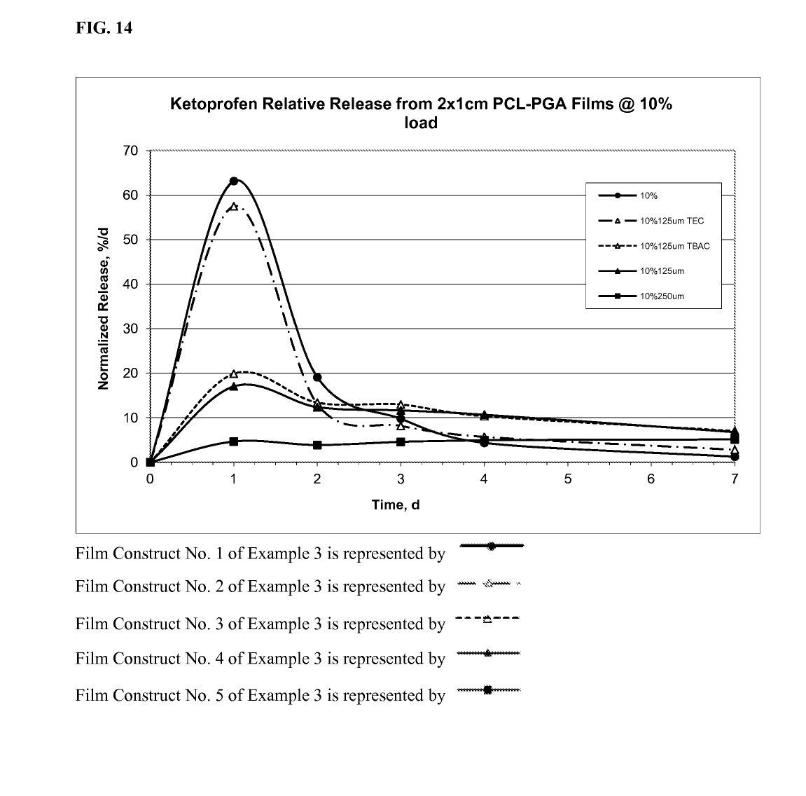

FIGS. 10 to 16 show a quick release of KTP (over a 4-day period) from the monolayer film consisting only of a carrier layer, while the KTP release is the slowest (up to 28 days) when the carrier layer is laminated between two barrier layers of identical thickness (250 um, Film Construct No. 5 above).

With respect to FIGS. 10-14:

Film Construct No. 1 is represented by [symbol]
Film Construct No. 2 is represented by [symbol]
Film Construct No. 3 is represented by [symbol]
Film Construct No. 4 is represented by [symbol]
Film Construct No. 5 is represented by

[symbol]

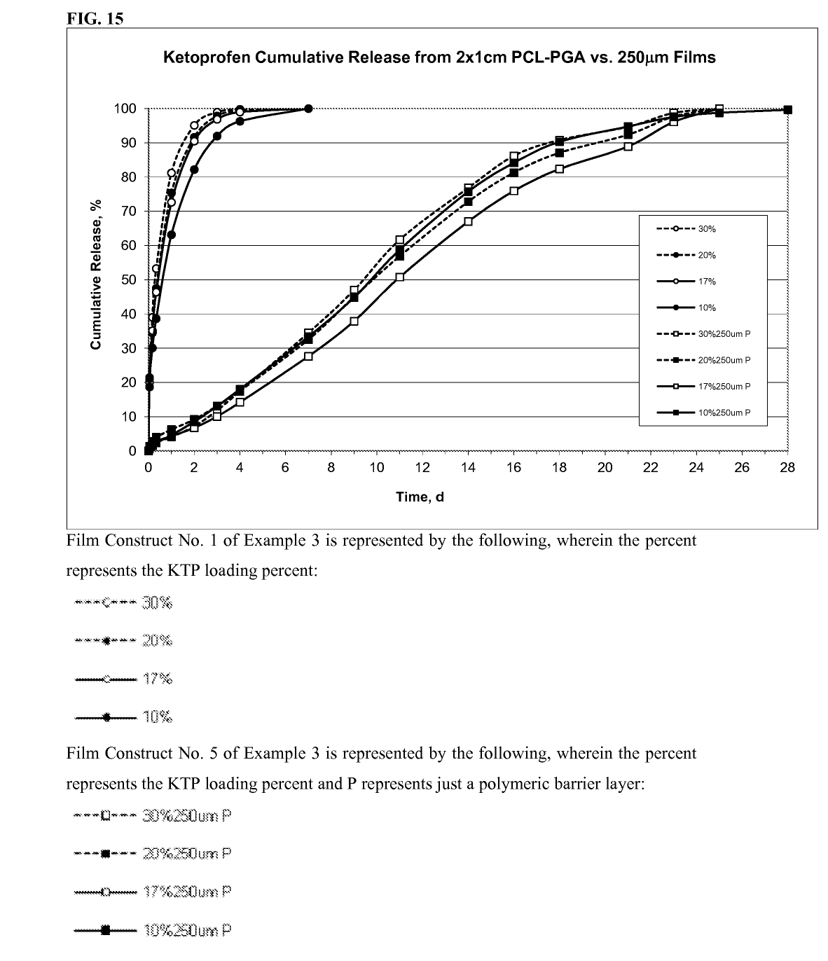

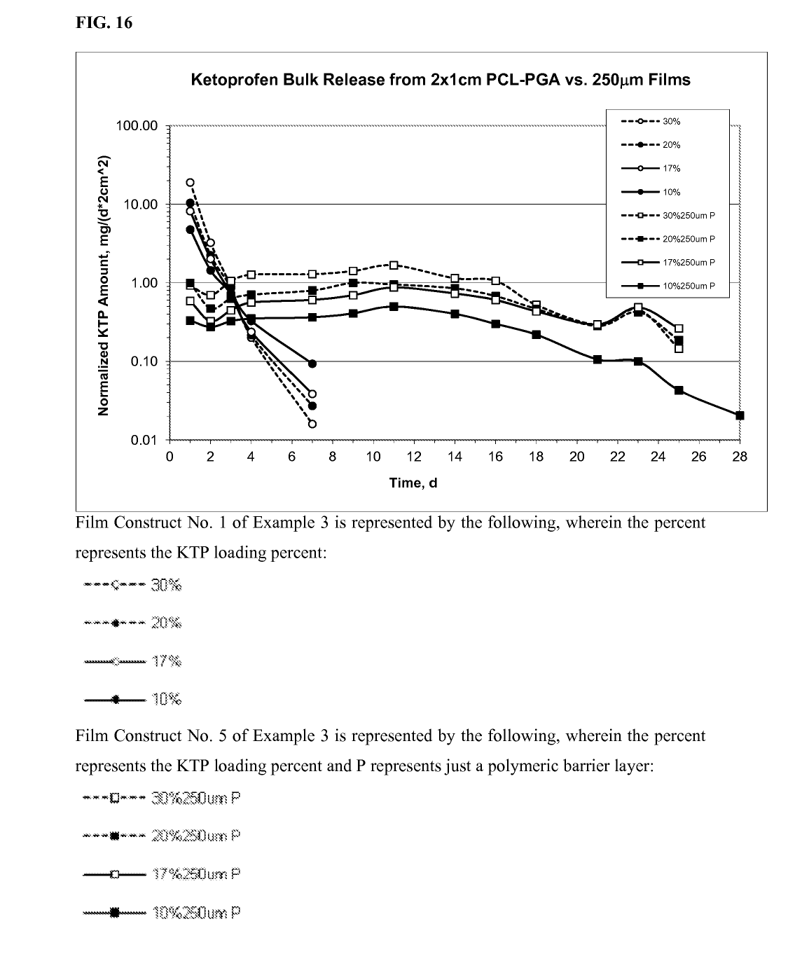

With respect to FIGS. 15-16:

Film Construct No. 1 is represented by the following, wherein the percent represents the KTP loading percent: [symbol]
30% [symbol]
20% [symbol]
17% [symbol]
10%
Film Construct No. 5 is represented by the following, wherein the percent represents the KTP loading percent and P represents just a polymeric barrier layer:

### 30% 250 um P

### 20% 250 um P

### 17% 250 um P

### 10% 250 um P

It was also observed that the release rate of KTP decreased as the thickness of the barrier layers increased, and for a given thickness of barrier layer, the addition of a plasticizer increased the release rate of KTP. Moreover, it was observed that a hydrophilic plasticizer had a greater effect on the release rate of KTP than a hydrophobic one.

### Example 4

### Lamination of ORC Layer onto a Polymeric Layer/Film

This example describes the method of laminating an oxidized regenerated cellulose (ORC) fabric onto a polymeric film containing an active (carrier layer as prepared in Example 1) or no active (barrier layer (see Example 2)) by compression molding. The ORC fabric, which was woven, used was an absorbable adhesion barrier sold under the trademark INTERCEED® (available from GYNECARE, a division of Ethicon, Inc., Somerville, N.J.), an absorbable hemostat sold under the trademark SURGICEL® ORIGINAL™ (available from Johnson & Johnson Wound Management, a division of Ethicon, Inc., Somerville, N.J.), and an absorbable hemostat sold under the trademark SURGICEL® NU-KNIT® (available from Johnson & Johnson Wound Management, a division of Ethicon, Inc., Somerville, N.J.), as shown in FIGS. 6, 7, and 8, respectively.

FIGS. 6, 7, and 8 show an ORC material laminated onto a polymeric barrier layer (no active). The lamination of a selected ORC fabric onto a polymeric film was performed in such a way that part of the ORC fibers were embedded into the polymeric film layer (the barrier layer) and part of these fibers remained exposed.

ORC-PCL/PGA 36/64 polymeric film constructs were prepared by compression molding under solvent-free conditions as described in Table 7 (see FIGS. 6, 7, and 8).

TABLE 7
Compression molding conditions for laminating ORC woven fabrics onto
PCL/PGA 36/64 polymeric films.

| | | Temperature, | |
|---|---|---|---|
| Step | Time, min | degrees Fahrenheit | Pressure, psi |
| 1 | Less than 1 | 266 | 0 |
| 2 | 5-6 | 266 | 10-12 |
| 3 | 8-10 | Less than 70 | 0 |

### Example 5

### Prevention of Tissue Constriction

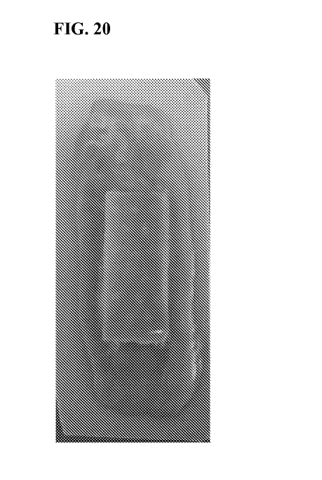

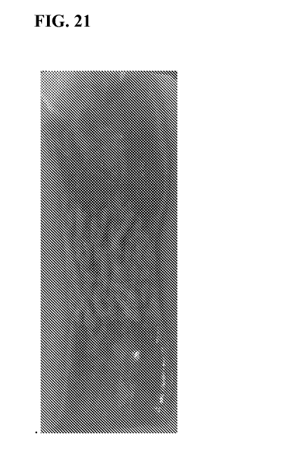

Above and beyond the localized delivery of actives, the ORC-polymeric film prepared as described in Example 4 has shown the capability of prevention of tissue constriction following the addition of cyanoacrylate sealant(s). Ex-vivo experiments on porcine GI tracts have demonstrated that an ORC-Polymeric Film comprising a PCL/PGA 36/64 barrier layer and an ORC layer (FIG. 20) prevents acute constriction of the tissue when compared to cyanoacrylate sealants alone (FIG. 21).

### Example 6

### Formulation and Preparation of Fully Encapsulated Multilayer Polymeric Films Containing a Temperature Sensitive Drug (Bimatoprost)

A sustained release drug dispensing device of cylindrical shape (FIG. 5) and comprised of two outer drug release controlling barrier layers each laminated to a middle drug-release-controlling barrier layer having a central inner void forming a reservoir for containing a drug-polymer formulation (carrier disk) which is defined by the internal surfaces of all the layers was manufactured as follows: first and second barrier layer materials were manufactured by compression molding commercially available 36/64 PCL/PGA polymer at 266 degrees Fahrenheit under 25,000 lbs of force using a 500 um thick mold to control the layer thickness. Next, a middle barrier layer was manufactured by compression molding commercially available 36/64 PCL/PGA polymer at 266 degrees Fahrenheit under 25,000 lbs of force using a 250 um thick mold to control the layer thickness. Next, first and second lower melting adhesive layers were manufactured by compression molding commercially available PCL polymer at 167 degrees Fahrenheit under 20,000 lbs of force using a 25 um thick mold to control the layer thickness. Next, the drug-containing layer (carrier layer) was manufactured by mixing 70 percent PCL and 30 percent bimatoprost at 152 degrees Fahrenheit followed by compression molding the mixture at 152 degrees Fahrenheit under 20,000 lbs of force using a 175 um thick mold to control the layer thickness. Next, 2 mm diameter holes were punched into the middle barrier layer and filled with a 2 mm diameter carrier disk that was punched from the drug-containing carrier layer.

Next, a stack was formed with the first barrier layer material followed by the first lower melting adhesive layer followed by the middle barrier layer containing a 2 mm diameter carrier disk from the drug containing carrier layer, followed by the second lower melting fusing layer, and then the second barrier layer material. The stack was then laminated together under 5,000 lbs of force and 140 degrees Fahrenheit using a 1.27 mm shim to control the height of the stack.

Next a 4 mm diameter sample was punched from the stack locating the 2 mm diameter carrier disk from the drug containing carrier layer concentric to the outer perimeter. Although not done in this experiment, a final laminating step can by performed under 5,000 lbs of force and 140 degrees Fahrenheit using a 1.27 mm shim to control the height of the stack.

### Example 7

### In-Vitro Release Studies of Temperature Sensitive Drug (Bimatoprost) from Fully Encapsulated Multilayer Polymeric Films

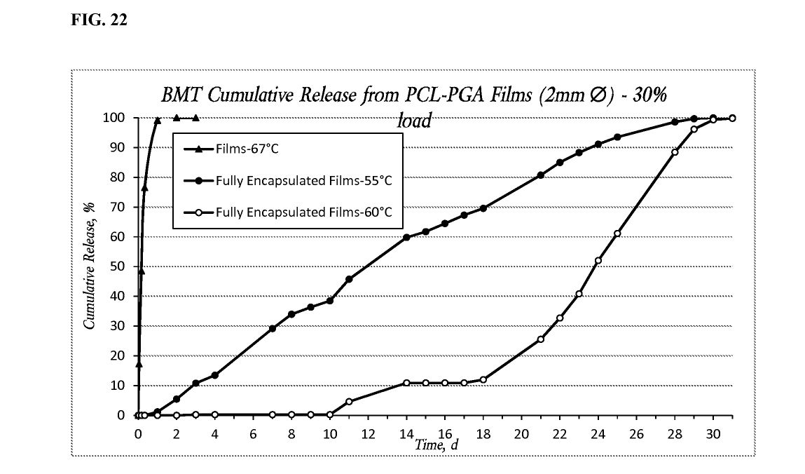

In Vitro release studies were performed using the fully encapsulated polymeric films (wherein the carrier discs are fully encapsulated within the surrounding barrier layers) containing bimatoprost as prepared in Example 6. Specifically, in vitro studies in PBS supplemented with 2 percent FBS at 37 degrees Celsius indicated that the final laminating temperature of the fully encapsulated disks allows for additional customization of the releases profiles as displayed in FIG. 22. More specifically, when laminating the multilayer construct, or fully encapsulated film, at 131 degrees Fahrenheit (55 degrees Celsius), a zero-order release of bimatoprost was observed. The same construct, when laminated at 140 degrees Fahrenheit (60 degrees Celsius), showed a period of no or undetectable release for the first 10 days, followed by a delayed sustained release of bimatoprost. Finally, a monolayer 2 mm diameter carrier film prepared by compression molding at 152 degrees Fahrenheit (67 degrees Celsius) displayed a release profile characterized by a bimatoprost burst (99 percent of the drug was released in the first 24 hours).

While not needed/done for this experiment, where there is a need for manufacturing selected formulations at lower temperatures, PCL can be substituted with PCL/PGA 90/10 in the carrier layer as well as for the adhesive layer. The processing temperature can then be decreased to 110 degrees Fahrenheit. The processing temperature can be further decreased to 100 degrees Fahrenheit provided that the PCL/PGA 90/10 has been heated beforehand at least at 118 degrees Fahrenheit.

### Example 8

### In-Vitro Release Studies of a Water Soluble Drug (5-Fluoro-Uracil (5-FU)) from Partially Encapsulated Multilayer Polymeric Films

In-Vitro release of 5-Fluoro-Uracil (5-FU) has been quantified over time from Partially Encapsulated Multilayer Polymeric films. A three-layer construction similar to the one shown in FIG. 3a (without the ORC layer 20) was prepared. Briefly, a carrier layer was prepared by admixing 113.72 mg of 5-FU (powder) with 266.8 mg of PCL/PGA 36/64 (powder) into a 33×33 mm mold, followed by compressing and heating under controlled conditions as described in Table 8.

TABLE 8
Compression molding conditions for preparing 5-FU polymeric
carrier films.

| | | Temperature, | |
|---|---|---|---|
| Step | Time, min | degrees Fahrenheit | Pressure, lbs |
| 1 | 1 | 252 | 10 |
| 2 | 3 | 252 | 1,000 |
| 3 | 5 | 252 | 20,000 |
| 4 | 30 | 70 | 20,000 |
| 5 | 1 | 70 | 0 |

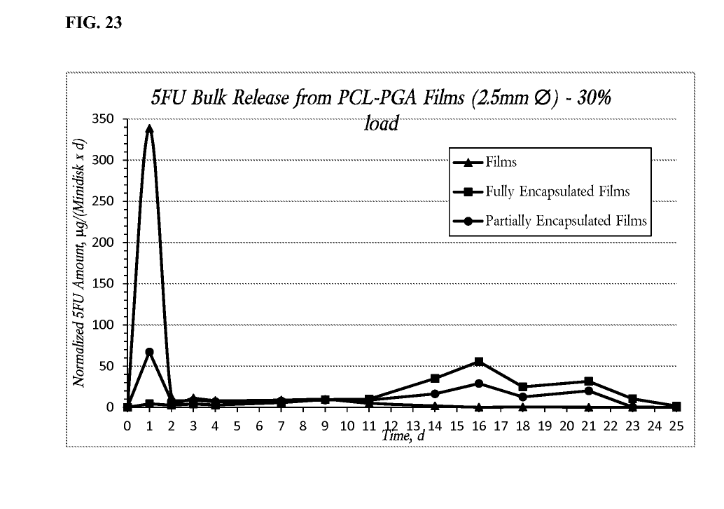

The nominal drug load in the carrier layer was 30 percent w/w. Two barrier layers were made of PCL/PGA (36/64) and prepared as described above in Example 2/Table 4. The carrier layer was subsequently laminated between the two barrier layers according to the process described in Example 2/Table 5. The partially encapsulated multilayer polymeric films were obtained by die cutting the 3-layer film. The resulting test articles, 2.5 mm diameter×1.25 mm high, were immersed in a PBS solution supplemented by 2 percent FBS onto a shaker in an incubator at 37 degrees Celsius. Release of 5-FU was quantified by HPLC-UV as follows: after filtration, aliquots were injected onto an Agilent 1100 column (50×4.6 mm×3 um with guard column). The 5-FU detection was performed via a UV Detector (Wavelength: 265 nm). The flow rate was 1.0 mL/min. The mobile phase was a mixture of TFA (0.1 percent) and Acetonitrile. In addition, a carrier disk was punched from the 5-FU loaded carrier layer and fully encapsulated into polymeric material as described in Example 7, without polymeric adhesive layers. Carrier disks were also obtained from the carrier layer (monolayer, see Example 1) alone. The analytical results presented in FIG. 23 showed a bimodal release profile of 5-FU partially encapsulated film that was characterized by a 2-day burst followed by a rest period of approximately 9 days then by a delayed sustained release starting around day 11. In addition FIG. 23 also displays the release profile (burst only) of 2.5 mm disks or films die-cut from the carrier layer alone (mono layer) as well as of fully encapsulated films prepared as described in Example 7 with no adhesive layers (delayed sustained release).

### Example 9

### In-Vitro Studies of ORC-Polymeric Film(s) Constructs in Rabbit Gastrointestinal (GI) Tract

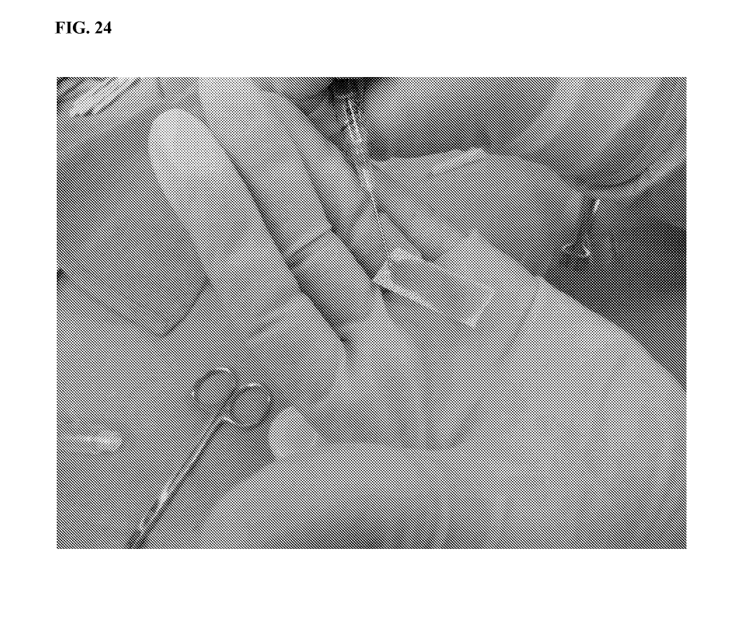

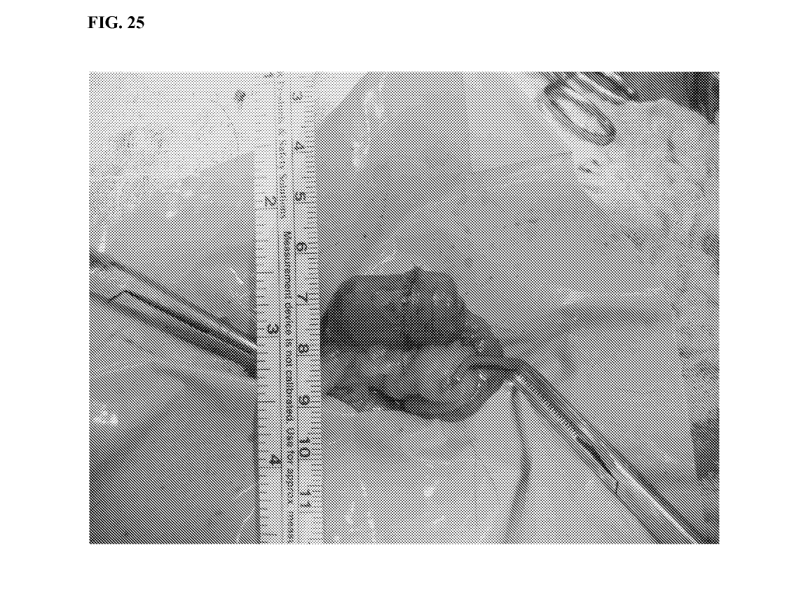

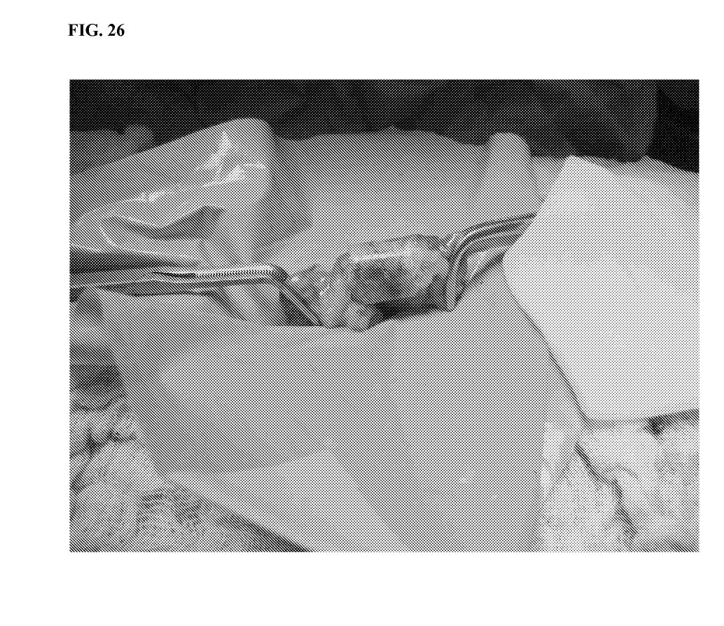

ORC-polymeric films were prepared as described in Example 4 (with no bioactive agent(s)) and implanted onto the rabbit GI tracts. It was found that after three (3) weeks the films had retained their integrity. Specifically, a radial transection of the rabbit GI tract was performed, and 2-octyl cyanoacrylate sealant was dispensed onto the ORC layer of the ORC-PCL/PGA films illustrated in FIGS. 6 and 18 as shown in FIG. 24. The ORC-PCL/PGA film with sealant was wrapped around the anastomosis line of the GI tract as an adjunct to the suture. See FIG. 25 illustrating the colon anastomosis (suture) in the rabbit and FIG. 26 illustrating the rabbit colon repaired with the ORC-PCL/PGA film as an adjunct to the suture. It was observed after three (3) weeks that the ORC-PCL/PGA film with sealant was still present on the rabbit GI tract, confirming that the ORC-PCL/PGA film indeed retains its integrity.

### Example 10

### In-Vitro Release Studies of Large Molecules

### Monoclonal Antibody to Human Necrosis Factor (Infliximab) from 3 Different Constructs

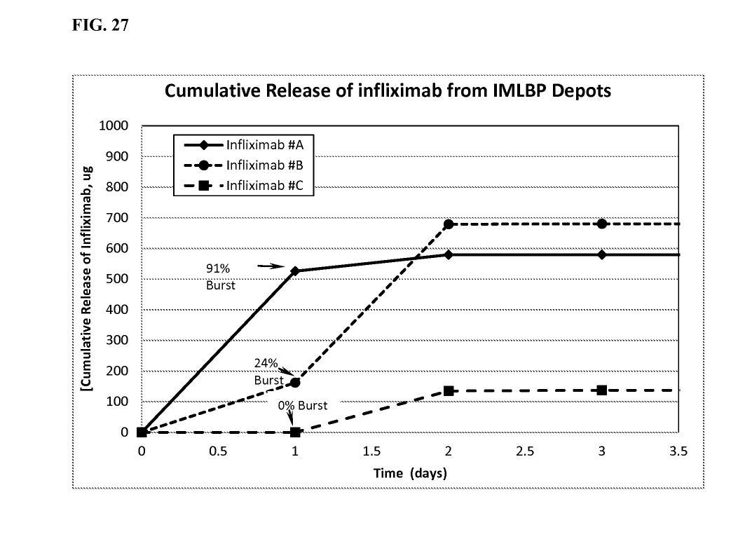

In Vitro release profiles of 3 selected constructs (FIG. 27) are described below:

### Construct A.

A monolayer (carrier layer) construction as shown in FIG. 1 (layer 30) was prepared. Briefly a carrier layer was prepared by admixing 5 mg of lyophilized infliximab, sold under the tradename REMICADE by Centocor Ortho Biotech, Inc., in Horsham, Pa., with 16 mg of PCL/PGA 90/10 powder into a 4×12 mm mold, followed by compressing at approximately 250 lbs and heating at 100 degrees Fahrenheit for 4 minutes (infliximab #A). As expected, Construct A resulted in a high burst release of infliximab.

### Construct B.

A modified construct of a partially encapsulated multilayer polymeric film as shown in FIG. 4a (without the ORC layer 40) was prepared to allow for a small burst release followed by sustained release of infliximab over days. Briefly, two 4×12 mm barrier layers were made of PCL/PGA (36/64) and prepared as described above in Example 2/Table 4. A carrier layer, such as that shown in FIG. 1 and described above for Construct A, was subsequently laminated between the two barrier layers according to the process described in Table 9.

TABLE 9
Compression molding conditions for laminating barrier layers onto carrier
layers.

| | | Temperature, | |
|---|---|---|---|
| Step | Time, min | degrees Fahrenheit | Pressure, lbs |
| 1 | 4 | 110 | 180 |
| 2 | 15 | 70 | 180 |
| 3 | 1 | 70 | 0 |

### Construct C.

A modified construct of a fully encapsulated multilayer polymeric film as shown in FIG. 5a was prepared to allow for a zero burst and delayed sustained release of infliximab. By modified, we mean that the construct was rectangular, not circular as shown in FIG. 5a. Briefly, a carrier layer “A”, rectangular in shape, was fully encapsulated, via compression molding as set forth in Table 9, within PCL/PGA 35/65 barrier layers, also rectangular in shape. These layers were subsequently locally sealed along the perimeter of the construct to maintain the integrity of the active, namely infliximab, and minimize the potential of degradation. Specifically, another compression molding step was performed wherein only 2 mm (wide) of each of the four (4) edges of the rectangular shaped construct were compression molder as set forth in Steps 1 and 5 of Table 8.

This design resulted in a zero burst at day 1 followed by relatively small release of the peptide within 3 days.

The identity and integrity of infliximab was confirmed using two methods: an ELISA method specific for the quantification of infliximab and a polyacrylamide SDS gel under denaturing and non-denaturing conditions.

The invention being thus described, it will be apparent that the same may be varied in many ways. Such variations are not to be regarded as a departure from the spirit and scope of the invention, and all such modifications as would be obvious to one skilled in the art are intended to be included within the scope of the following claims.

We claim:

1. A solvent-free sustained release device comprising:
A carrier layer having an upper surface and a lower surface, the carrier layer comprising a biocompatible, biodegradable polymer and a bioactive agent; and
A first oxidized regenerated cellulose layer laminated to the upper surface of the carrier layer.

2. The device of claim 1 further comprising a second oxidized regenerated cellulose layer laminated to the lower surface of the carrier layer.

3. The device of claim 1 wherein the second oxidized regenerated cellulose layer is dry, partially neutralized to a pH of from about 5 to about 7.

4. The device of claim 3 wherein the second oxidized regenerated cellulose layer further comprises a bioabsorbable alpha-cyanoacrylate adhesive composition.

5. A solvent-free sustained release device comprising:
A carrier layer having an upper surface and a lower surface, the carrier layer comprising a biocompatible, biodegradable polymer and a bioactive agent;
A first barrier layer having an upper surface and a lower surface, the first barrier layer comprising a biocompatible, biodegradable polymer wherein the lower surface of the first barrier layer is laminated to the upper surface of the carrier layer;
A second barrier layer having an upper surface and a lower surface, the second barrier layer comprising a biocompatible, biodegradable polymer wherein the upper surface of the second barrier layer is laminated to the lower surface of the carrier layer; and
A first oxidized regenerated cellulose layer laminated to the upper surface of the first barrier layer.

6. The device of claim 5 further comprising a second oxidized regenerated cellulose layer laminated to the lower surface of the second barrier layer.

7. The device of claim 5 wherein the second oxidized regenerated cellulose layer is dry, partially neutralized to a pH of from about 5 to about 7.

8. The device of claim 7 further wherein the second oxidized regenerated cellulose layer further comprises a bioabsorbable alpha-cyanoacrylate adhesive composition.

9. A solvent-free sustained release device comprising:
A first barrier layer having an upper surface and a lower surface;
A first adhesive layer having an upper surface and a lower surface;
A middle barrier layer, having an upper surface and a lower surface, comprising a hole;
A carrier disk comprising a bioactive agent and disposed in the hole of the middle barrier layer;
A second adhesive layer having an upper surface and a lower surface;
A second barrier layer having an upper surface and a lower surface; and
wherein the lower surface of the first barrier layer is laminated to the upper surface of the first adhesive layer, the lower surface of the first adhesive layer is laminated to the upper surface of the middle barrier layer, the lower surface of the middle barrier layer is laminated to the upper surface of the second adhesive layer, and the lower surface of the second adhesive layer is laminated to the upper surface of the second barrier layer; and
wherein the first and second barrier layers, the first and second adhesive layers, the middle barrier layer, and the carrier disk comprise a biocompatible, biodegradable polymer.
